(19) United States
(12) Patent Application Publication
(10) Pub. No.: US 2011/0118504 A1
(43) Pub. Date: May 19, 2011

(54) THERMAL SALT SPLITTING OF AMMONIUM CARBOXYLATES

(75) Inventors: Thomas Haas, Muenster (DE); Thomas Tacke, Alzenau (DE); Alexander Schraven, Issum (DE); Hans-Joachim Schleef, Havixbeck (DE); Oliver Zehnacker, Recklinghausen (DE)

(73) Assignee: EVONIK ROEHM GMBH, DARMSTADT (DE)

(21) Appl. No.: 13/054,002

(22) PCT Filed: May 27, 2009
(86) PCT No.: PCT/EP09/56402

§ 371 Date: Jan. 13, 2011

(30) Foreign Application Priority Data
Jul. 15, 2008 (DE) 102008040415.2

(51) Int. Cl. C07C 51/347

(52) U.S. Cl. 562/579

(57) ABSTRACT

The present invention relates to a process for preparing hydroxycarboxylic acids, preferably α- and β-hydroxycarboxylic acids, from ammonium carboxylates of the general formula

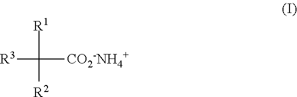

in which R¹, R² and R³ are each independently H, OH, (C₁-C₆)-alkyl optionally substituted by a hydroxyl group, (C₁-C₆)-alkenyl optionally substituted by a hydroxyl group, (C₁-C₆)-alkoxy optionally substituted by a hydroxyl group, (C₁-C₆) -alkylthio-(C₁-C₆)-alkyl optionally substituted by a hydroxyl group, (C₆-C₁₀)-aryl optionally substituted by a hydroxyl group, (C₇-C₁₂)-aralkyl optionally substituted by a hydroxyl group, (C₃-C₅)-heteroaryl optionally substituted by a hydroxyl group, with the proviso that at least one hydroxyl group is present in at least one R¹, R² and R³ radical,
preferably R¹═H, CH₃, CH₂CH₃, C₆H₅, (CH₂)₂SCH₃ and R²═H, CH₃ and R³═OH,
equally preferably R¹═CH₂OH, CHOHCH₃ and R²═R³═H, CH₃,
more preferably R¹═R²═CH₃ and R³═OH,
equally more preferably R¹═CH₂OH, R²═CH₃ and R³═H, comprising the following step:
heating an aqueous starting solution comprising the ammonium carboxylate to form, by thermal decomposition of the ammonium carboxylate, the hydroxycarboxylic acid and ammonia, and simultaneously to remove at least a portion of the free water and of the ammonia formed from the solution and thus to obtain a product fraction comprising the hydroxycarboxylic acid,
characterized in that
the content of the ammonium salt in the starting solution is less than 60% by weight, the thermal decomposition of the ammonium salt and the removal of the free water and of the ammonia formed are effected in one process step, the conversion of the ammonium salt being more than 20 mol %, preferably more than 30 mol %, more preferably more than 50 mol %, especially preferably more than 75 mol %, very especially preferably more than 90 mol % and especially more than 95 mol %, and no ether, alcohol or hydrocarbon is used as an entraining agent.

The present invention relates to a process for preparing hydroxycarboxylic acids from ammonium carboxylates of the general formula

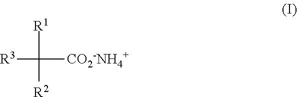

in which R¹, R² and R³ are each independently H, OH, (C₁-C₆)-alkyl optionally substituted by a hydroxyl group, (C₁-C₆)-alkenyl optionally substituted by a hydroxyl group, (C₁-C₆)-alkoxy optionally substituted by a hydroxyl group, (C₁-C₆) -alkylthio-(C₁-C₆) -alkyl optionally substituted by a hydroxyl group, (C₆-C₁₀)-aryl optionally substituted by a hydroxyl group, (C₇-C₁₂)-aralkyl optionally substituted by a hydroxyl group, (C₃-C₅)-heteroaryl optionally substituted by a hydroxyl group, with the proviso that at least one hydroxyl group is present in at least one R¹, R² and R³ radical, by heating an aqueous starting solution comprising the ammonium carboxylate to form, by thermal decomposition of the ammonium carboxylate, the hydroxycarboxylic acid and ammonia, and simultaneously to remove at least a portion of the free water and of the ammonia formed from the solution and thus to obtain a product fraction comprising the hydroxycarboxylic acid.

Hydroxycarboxylic acids of the general formula

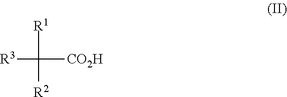

for example glycolic acid, lactic acid or 2-hydroxyisobutyric acid, are important starting materials in the field of pharmaceutical chemistry, of agrochemistry and of polymer chemistry, and are used for the synthesis of intermediates used on the industrial scale, for example acrylic acid derivatives, and are additionally used as food and animal feed additives. Hydroxycarboxylic acids can be effected by chemical syntheses or biotechnology methods such as the fermentation of sugars or starch using microorganisms, or the enzymatic hydrolysis of carbonitriles.

When the substituents R¹, R² and R³ in the general formula (I) are different from one another and are not CO₂H, there exist two optically active forms (enantiomers) of the compound. While only one racemate of the two enantiomers is often obtained in chemical syntheses, it is often possible to achieve high excesses of one enantiomer in biotechnology methods. The enantiomer formed preferentially can be selected here via a suitable selection of the microorganism or enzyme. In biotechnology methods, the carboxylic acid is frequently obtained as an aqueous solution of an ammonium carboxylate. The content of the ammonium carboxylate in the fermentation broth or the reaction solution of an enzymatic reaction depends on the process used, but in many cases does not exceed 10% by weight and is frequently even much lower (EP 1 466 984 A1, U.S. Pat. No. 6,937,155, U.S. Pat. No. 7,198,927 B2).

The prior art discloses a series of processes for preparing the free hydroxycarboxylic acids from an aqueous solution of the corresponding ammonium carboxylate, for example cationic or anionic ion exchange chromatography, electrodialysis, extraction with reactive solvents or acidification of the fermentation broth with mineral acids and subsequent isolation of the carboxylic acid by concentration, crystallization or distillation (Joglekar et al. Separation and Purification Technology, 2006, 52, 1-17). Many of these methods have crucial disadvantages with regard to the preparation of hydroxycarboxylic acids on the industrial scale. Some of the processes are very costly, especially with regard to the relatively low concentrations of the ammonium carboxylate in the solution obtained from a biotechnology method, some of them require expensive and fault-prone apparatus and/or generate, through the use of additional chemicals, molar amounts of by-products which either have to be disposed of or recycled in a complicated manner. For example, when the ammonium carboxylate is acidified with a mineral acid or in the case of ion exchange chromatography, molar amounts of a mineral salt are formed, which causes additional disposal costs.

Another approach to obtaining free hydroxycarboxylic acids from their corresponding ammonium carboxylates is the thermal decomposition of the ammonium carboxylate to the free acid and ammonia according to equation (i).

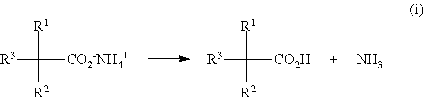

U.S. Pat. No. 6 291 708 B1 describes a process in which an aqueous solution of an ammonium salt is mixed with a suitable alcohol and this alcohol-water mixture is then heated under elevated pressure in order to decompose the ammonium salt thermally to the free acid and ammonia. At the same time, a suitable gas is contacted as an entraining agent with the alcohol-water mixture, so as to drive out a gaseous product stream comprising ammonia, water and a portion of the alcohol, while at least 10% of the alcohol remains in the liquid phase and reacts with the free acid to form the corresponding ester. The disadvantages of this process include the need for additional chemicals (alcohol and a gas as an entraining agent) and the partial conversion of the free carboxylic acid formed to the ester, which in turn has to be hydrolyzed in order to obtain the free carboxylic acid.

US 2003/0029711 A1 describes a process for obtaining organic acids, inter alia from aqueous solutions of the ammonium salts with addition of a hydrocarbon as an entraining agent. Heating of the mixture affords a gaseous product stream which comprises an azeotrope consisting of the organic acid and the entraining agent. In order to isolate the acid from this product stream, further steps such as condensation and additional distillations have to be carried out. Furthermore, this process also requires the addition of additional chemicals (entraining agents), which makes the process significantly more costly, specifically for application on the industrial scale.

EP 0 884 300 A1 describes a two-stage process for obtaining α-hydroxycarboxylic acids from the corresponding ammonium salts, in which, in a first step, an aqueous solution of the ammonium salt is heated either as such or in a suitable organic solvent, for example xylene, toluene or anisole, so as to form low molecular weight poly-α-hydroxycarboxylic acids and, in addition to the free water, also to remove a portion of the water formed by the condensation of monomeric α-hydroxycarboxylic acid to poly-α-hydroxycarboxylic acid, and ammonia. In a second process step, thereafter, the readdition of water and the heating of the resulting aqueous solution is necessary in order to hydrolyze the poly-α-hydroxycarboxylic acid to the monomeric α-hydroxycarboxylic acid. As well as the additional process step, further disadvantages of this process are the addition of an entraining agent (the azeotropic reagent) and the greatly reduced pressure which is required when no azeotropic reagent is added to the aqueous solution (typically less than 0.002*10⁵ Pa when the organic entraining agent is dispensed with), and the high required starting concentration of the ammonium carboxylate under aqueous solution (content more than 80% by weight when no entraining agent is employed).

A related process is described in WO 2006/069129 A1. Here, in a first step, the free water is very substantially removed from an aqueous solution of the ammonium carboxylate and the anhydrous ammonium carboxylate is thus obtained. This is then heated to 100 to 140° C. in a separate process step under reduced pressure, in which the thermal decomposition of the salt takes place, the ammonia formed is removed under reduced pressure, and a product mixture of poly-hydroxy acids, oligomers of the hydroxycarboxylic acids, oligomers of the ammonium salts and unconverted ammonium carboxylate is thus obtained. This product mixture subsequently has to be admixed with water in a further process step and heated for hydrolysis. In this process too, the preparation of a very substantially anhydrous salt is necessary, which can be decomposed thermally only in a separate process step. Moreover, yet a further separate process step for hydrolysis is needed.

WO 00/59847 describes a process for preparing hydroxycarboxylic acids from aqueous solutions of their ammonium salts. The process described there also requires a separate process step for concentration of the aqueous ammonium salt solution, since the concentration of the ammonium salt in the aqueous solution for the aqueous salt splitting must be more than 60% by weight, and a further separate process step for thermal decomposition of the ammonium salt, which additionally also requires the use of an entraining agent to remove the ammonia formed.

Problems which additionally occur in many of the literature methods are firstly the formation of considerable amounts of hydroxycarboxamide through condensation of the carboxylic acid formed in the reaction with the ammonia which is likewise released according to equation (ii):

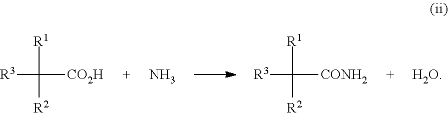

In addition, in the case of reaction of ammonium salts of optically active hydroxycarboxylic acids, specifically in the presence of strong acids or bases and at elevated temperatures, there is the risk of epimerization of the stereocentre which, according to the reaction conditions, may lead to the complete loss of the stereo information to form a racemic mixture.

It was therefore an object of the present invention to provide a process for obtaining free hydroxycarboxylic acids from aqueous solutions of their ammonium salts, in which there is no need for any concentration of the aqueous solution in a separate process step, and the thermal decomposition of the ammonium salt and the removal of the ammonia formed and of the free water from an aqueous solution can instead be effected in a single process step without addition of an organic solvent as an entraining agent.

It has now been found that, surprisingly, hydroxycarboxylic acids can be obtained by thermal salt splitting of aqueous solutions of their ammonium salts, in which the content of the ammonium salt is less than 60% by weight, by heating the aqueous solution, which at the same time allows at least a portion of the free water and of the ammonia formed to be removed, without any need to use an organic solvent or inert gas as an entraining agent.

The present invention therefore provides a process for preparing hydroxycarboxylic acids, preferably α- and β-hydroxycarboxylic acids, from ammonium carboxylates of the general formula

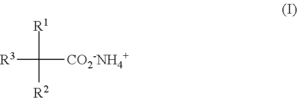

in which R¹, R² and R³ are each independently H, OH, (C₁-C₆)-alkyl optionally substituted by a hydroxyl group, (C₁-C₆)-alkenyl optionally substituted by a hydroxyl group, (C₁-C₆)-alkoxy optionally substituted by a hydroxyl group, (C₁-C₆) -alkylthio-(C₁-C₆) -alkyl optionally substituted by a hydroxyl group, (C₆⁻C₁₀)-aryl optionally substituted by a hydroxyl group, (C₇-C₁₂)-aralkyl optionally substituted by a hydroxyl group, (C₃-C₅)-heteroaryl optionally substituted by a hydroxyl group, with the proviso that at least one hydroxyl group is present in at least one R¹, R² and R³ radical,
comprising the following step:

heating an aqueous starting solution comprising the ammonium carboxylate to form, by thermal decomposition of the ammonium carboxylate, the hydroxycarboxylic acid and ammonia, and simultaneously to remove at least a portion of the free water and of the ammonia formed from the solution and thus to obtain a product fraction comprising the hydroxycarboxylic acid,

characterized in that

the content of the ammonium salt in the starting solution is less than 60% by weight, the thermal decomposition of the ammonium salt and the removal of the free water and of the ammonia formed are effected in one process step, the conversion of the ammonium salt being more than 20 mol %, preferably more than 30 mol %, more preferably more than 50 mol %, especially preferably more than 75 mol %, very especially preferably more than 90 mol % and especially more than 95 mol %, and no ether, alcohol or hydrocarbon is used as an entraining agent.

There is preferably no further concentration of the starting solution before the thermal salt splitting.

Particular preference is given to employing the process to prepare the α-hydroxycarboxylic acids glycolic acid (R¹═R²═H; R³═OH), lactic acid (R¹═CH₃; R²═H; R³═OH), citric acid (R¹═R²═CH₂COOH; R³═OH), tartaric acid (R¹═CHOHCOOH; R²═H; R³═OH), 2-hydroxyisobutyric acid (R¹═R²═CH₃; R³═OH), 2-hydroxy-2-phenylpropanoic acid (R¹═CH₃; R²═Ph; R³═OH) and 4-methylthiobutyric acid (R¹═CH₂CH₂SCH₃; R²═H; R³═OH), particularly preference being given to 2-hydroxyisobutyric acid, and to prepare the β-hydroxycarboxylic acids 3-hydroxypropionic acid (R¹═CH₂OH; R²═H; R³═H), 3-hydroxybutyric acid (R¹═CH₂OHCH₃; R²═H; R³═H), 3-hydroxyvaleric acid (R¹═CH₂OHCH₂CH₃; R²═H; R³═H), 3-hydroxyhexanoic acid (R¹═CH₂OHCH₂CH₂CH₃; R²═H; R³═H), 3-hydroxyheptanoic acid (R¹═CH₂OHCH₂CH₂CH₂CH₃; R²═H; R³═H), 3-hydroxyoctanoic acid (R¹═CH₂OHCH₂CH₂CH₂CH₂CH₃; R²═H; R³═H) and 3-hydroxyisobutyric acid (R¹═CH₂OH; R²═CH₃; R³═H), particular preference being given to 3-hydroxyisobutyric acid.

In the context of the invention, “free water” means the water in the aqueous solution utilized as a solvent, in contrast to the water which could be formed in principle by condensation of the hydroxycarboxylic acids formed to poly-hydroxycarboxylic acids. One advantage of the present invention is that, in contrast to other processes, the ammonium salt of the hydroxycarboxylic acid, in the course of thermal salt splitting, need not first be converted to a large degree to (low molecular weight) poly-hydroxycarboxylic acids, from which the free hydroxycarboxylic acid can only be obtained by hydrolysis in a separate process step.

The method of heating depends on the apparatus/plant used and can be effected, for example, by means of a heating bath, a temperature-controllable reactor jacket or by contacting the starting solution with a heated gas stream. Preference is given to using apparatus with short residence times and large surface area, for example thin-film evaporators, short-path evaporators, falling-film evaporators. Depending on the pressure used, the temperature is selected such that the thermal salt splitting takes place and the formation of by-products such as carboxamides is minimized. Preferably, at least a portion of the free water and of the ammonia formed during the reaction is removed by distillation simultaneously. Suitable temperature and pressure ranges can be determined by a person skilled in the art, as can the necessary duration of the thermal treatment, for example by monitoring the amount of ammonia formed or the temperature profile of the reaction solution.

In a preferred embodiment, the temperature of the reaction solution is 70 to 300° C., preferably 80 to 250° C., especially 100 to 220° C. and more preferably 120 to 200° C.

In a further preferred embodiment, the heating of the aqueous starting solution comprising the ammonium carboxylate is performed under reduced pressure. In the context of the invention, a reduced pressure here means a pressure of less than 1×10⁵ Pa, preferably less than 0.9×10⁵ Pa and more preferably less than 0.8×10⁵ Pa and especially less than 0.7×10⁵ Pa.

Preference is given to selecting a combination of pressure, temperature and apparatus such that short residence times of the aqueous starting solution in the reaction apparatus are achieved.

In the context of the invention, entraining agents are both organic solvents which form an azeotrope with water or a component formed in the course of thermal salt splitting, and inert gases or vapours of the organic solvent which are used to drive out the ammonia formed and/or the water vapour (carrier gases). It is preferred in the context of the invention that no organic solvent or organic amine is used as an entraining agent or extractant. It is further preferred that no inert gas is used as an entraining agent to remove the ammonia and the water.

In a preferred embodiment, in contrast, air can be used as the carrier gas.

In a preferred embodiment, the concentration of the ammonium salt in the starting solution is less than 50% by weight, preferably less than 30% by weight, especially less than 20% by weight and more preferably less than 15% by weight.

The aqueous starting solution used may be a fermentation broth or the reaction solution of an enzymatic reaction to prepare the ammonium hydroxycarboxylate solution, which can optionally be partially purified before use in the process according to the invention. Processes for partial purification of fermentation broths are known to those skilled in the art and include, for example, filtration or centrifugation to remove the cell material. In this case, the starting solution may contain traces of organic solvent as a result of the fermentation process, but no organic solvent is added to the aqueous solution as an entraining agent or extractant. In the context of the invention, traces of organic solvents refer to the organic solvents which possibly form as by-products in the fermentation process (for example ethanol), whose proportion in the triggering is preferably less than 10 mol %, more preferably less than 5 mol %, especially preferably less than 2 mol % and especially less than 1 mol %, based on the amount of the ammonium carboxylate.

In addition, the starting solution can also be obtained from other sources, for example by degradation of polymers such as polylactide.

A further important aspect of the invention is that the proportion of hydroxycarboxamide in the product fraction is less than 25 mol %, preferably less than 15 mol %, especially less than 7.5 mol % and more preferably less than 1 mol %, based on the total amount of the hydroxycarboxylic acid derivatives. In the context of the invention, hydroxycarboxylic acid derivatives are understood to mean the free hydroxycarboxylic acid, oligo- and polyhydroxycarboxylic acids, the ammonium salt of the hydroxycarboxylic acid, and the hydroxycarboxamide.

In a preferred embodiment, the content of the ammonium salt during the overall process (i.e. in the starting solution, the reaction solution during the thermal salt splitting and the resulting product fraction) is less than 60% by weight, preferably less than 50% by weight, more preferably less than 30% by weight, especially less than 20% by weight and especially preferably less than 15% by weight. When R¹, R² and R³ are different from one another and are not COOH, the degree of epimerization of the resulting free hydroxycarboxylic acid, in a preferred embodiment, is less than 50%, preferably less than 25%, more preferably less than 10% and especially less than 5%, based on the enantiomeric excess of the ammonium carboxylate used.

The resulting product fraction can be converted without further purification to conversion products. Preference is given in the context of the invention, for example, to the dehydration of α- and β-hydroxycarboxylic acids to acrylic acid derivatives, where hydroxycarboxylic acids of the general formula (II), where α-hydroxycarboxylic acids where R¹═(C₁-C₆)-alkyl or (C₇-C₁₂)-aralkyl and R²═H, (C₁-C₆)-alkyl or (C₇-C₁₂)-aralkyl and R³═OH, and 3-hydroxycarboxylic acids where R¹═(C₁-C₆)-alkyl-OH or (C₇-C₁₂)-aralkyl-OH, and R² and R³ are the same or different and are each independently H, (C₁-C₆)-alkyl or (C₇-C₁₂)-aralkyl. A series of processes for dehydrating α- and β-hydroxycarboxylic acids to acrylic acid derivatives is known to those skilled in the art; they are described, for example, in PCT/EP2007/055394, U.S. Pat. No. 3,666,805 and U.S. Pat. No. 5,225,594.

The process according to the invention may further comprise one or more subsequent steps for purification and isolation of the hydroxycarboxylic acids from the product fraction. Suitable process steps include concentration, crystallization, ion exchange chromatography, electrodialysis, extraction and with reactive and also with inert solvents, and purification by esterification of the hydroxycarboxylic acid with suitable alcohols, subsequent distillation of the resulting ester and subsequent hydrolysis of the ester to the free acid, and combinations of these steps. By-products present in the product fraction can be removed before or after the isolation of the free hydroxycarboxylic acid formed in the thermal salt splitting, or be converted to the hydroxycarboxylic acid, for example by enzymatic or chemical hydrolysis of hydroxycarboxamides and oligo- or polyhydroxy-carboxylic acids. Since the product fraction, owing to the thermal salt splitting, contains significantly less ammonium salt and water than the starting solution, the amount of chemicals required in these subsequent optional process steps and the amount of waste obtained (for example of mineral salts in the case of acidic workup) is significantly lower than in the case of purification and isolation from the starting solution which has not been treated thermally by the process according to the invention beforehand.

### EXAMPLES

### Example 1

### Thermal Cleavage of Ammonium 2-hydroxyisobutyrate, Inventive

A round-bottom flask with Liebig condenser, to which a vacuum pump had been attached via a wash bottle, was initially charged with 20.53 g of an about 11% by weight aqueous ammonium 2-hydroxyisobutyrate solution (A-2HIBA). The solution was heated in an oil bath heated to 140° C. with stirring and, under reduced pressure (p=0.5×10⁵ Pa), the free water was distilled off with simultaneous thermal salt splitting. During the distillation, the mass in the round-bottom flask decreased to 2.86 g. After 180 minutes, the reaction was terminated. The ammonia concentration was determined by means of a Kjeldahl analysis. About 49% of the mass of ammonia introduced at the start (0.32 g) was detectable in the round-bottom flask. The rest of the ammonia was detectable in the distillate and the wash bottle. By means of an HPLC analysis, it was possible to detect the 2-hydroxyisobutyric acid concentration (as the free acid and as the salt) in the round-bottom flask. From this, it was possible to determine, with the aid of a stoichiometric assessment, the ratio between the free acid and the salt. About 48 mol % of the 2-hydroxyisobutyric acid introduced before the start of the experiment were present as the salt, and about 51 mol % as the free acid. Amides were detectable only in traces. Both the conversion and the yield of the free acid were about 51 mol %.

### Example 2

### Thermal Splitting of Ammonium 2 Hydroxyisobutyrate, Inventive

A round-bottom flask with Liebig condenser, to which a vacuum pump had been attached via a wash bottle, was initially charged with 20.03 g of an about 11% by weight aqueous ammonium 2-hydroxyisobutyrate solution (A-2HIBA). The solution was heated in an oil bath heated to 160° C. with stirring and, under reduced pressure (p=0.8×10⁵ Pa), the free water was distilled off with simultaneous thermal salt splitting. During the distillation, the mass in the round-bottom flask decreased to 2.51 g. After 180 minutes, the reaction was terminated. The ammonia concentration was determined by means of a Kjeldahl analysis. About 44% of the mass of ammonia introduced at the start (0.32 g) was detectable in the round-bottom flask. The rest of the ammonia was detectable in the distillate and the wash bottle. By means of an HPLC analysis, it was possible to detect the 2-hydroxyisobutyric acid concentration (as the free acid and as the salt) in the round-bottom flask. From this, it was possible to determine, with the aid of a stoichiometric assessment, the ratio between the free acid and the salt. About 42 mol % of the 2-HIBA introduced before the start of the experiment were present as the salt, and about 51 mol % as the free acid. About 5 mol % of the ammonium 2-hydroxyisobutyrate introduced before the start of the experiment reacted to give the undesired amide. The conversion of the salt introduced in this experiment was approx. 56 mol %, and the yield of free acid was about 51 mol %.

### Example 3

### Thermal Splitting of a Concentrated Ammonium 2-hydroxyisobutyrate Solution, Noninventive

A concentrated ammonium 2-hydroxyisobutyrate solution was prepared by weighing in 2-hydroxyisobutyric acid (2-HIBA), ammonium hydroxide and water. To this end, approx. 35.9 g of 2-hydroxyisobutyric acid, 21.6 g of ammonium hydroxide and 6.1 g of water were mixed with one another with constant stirring in a beaker. This established a pH of 7.5. This solution corresponds to an approx. 65% by weight ammonium 2-hydroxyisobutyrate solution.

The following step is based on the reactive evaporation of WO 00/59847: about 10.8 g of this solution were introduced into a round-bottom flask and heated in an oil bath. The temperature in the oil bath was adjusted to 180° C. and kept constant. A wash bottle and a vacuum pump were attached via a Liebig condenser. The system pressure was adjusted to 0.05×10⁵ Pa and kept constant. The experiment was terminated after 10 min and the remaining solution in the round-bottom flask was analyzed by means of HPLC. It was found that significant amounts of the amide had formed under these reaction conditions. Approx. 9% of the 2-hydroxyisobutyric acid analyzed (as the free acid, as the salt and as the amide) was present as the amide.

1. A process for preparing a hydroxycarboxylic acid from an ammonium carboxylate of the general formula 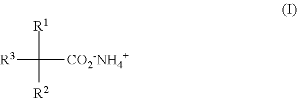
in which R¹, R² and R³ are each independently
H,
OH,
(C₁-C₆)-alkyl optionally substituted by a hydroxyl group,
(C₁-C₆)-alkenyl optionally substituted by a hydroxyl group,
(C₁-C₆)-alkoxy optionally substituted by a hydroxyl group,
(C₁-C₆)-alkylthio-(C₁-C₆)-alkyl optionally substituted by a hydroxyl group,
(C₆-C₁₀)-aryl optionally substituted by a hydroxyl group,
(C₇-C₁₂)-aralkyl optionally substituted by a hydroxyl group, or
(C₃-C₅)-heteroaryl optionally substituted by a hydroxyl group,
with the proviso that at least one hydroxyl group is present in at least one R¹, R² and R³ radical,
comprising:
heating an aqueous starting solution comprising the ammonium carboxylate to form, by thermal decomposition of the ammonium carboxylate, the hydroxycarboxylic acid and ammonia, and simultaneously to remove at least a portion of free water and of the ammonia formed from a reacting solution and thus to obtain a product fraction comprising the hydroxycarboxylic acid,
wherein
the content of ammonium salt in the starting solution is less than 60% by weight,
the thermal decomposition of the ammonium salt and the removal of the free water and of the ammonia formed are effected in one process step, and
the conversion of the ammonium salt being more than 20 mol %, and no ether, alcohol or hydrocarbon is used as an entraining agent.

2. The process according to claim 1,
wherein
the temperature of the reaction reacting solution is 70 to 300° C.

3. The process according to claim 1,
wherein
the heating of the aqueous starting solution comprising the ammonium carboxylate is performed under reduced pressure.

4. The process according to claim 1,
wherein
no organic solvent is used as an entraining agent.

5. The process according to claim 1,
wherein
no inert gas is used as an entraining agent to remove the ammonia and the free water.

6. The process according to claim 1,
wherein
a concentration of the ammonium salt in the starting solution is less than 50% by weight.

7. The process according to claim 1,
wherein
the aqueous starting solution is a fermentation broth or a reaction solution of an enzymatic reaction to prepare the ammonium hydroxycarboxylate, which may optionally have been partially purified beforehand.

8. The process according to claim 7, wherein the starting solution may contain traces of organic solvent as a result of the fermentation process, but no organic solvent is added as an entraining agent.

9. The process according to claim 1,
wherein
a proportion of hydroxycarboxamide in the product fraction is less than 25 mol % based on the total amount of the hydroxycarboxylic acid derivatives.

10. The process according to claim 1,
wherein
the ammonium salt during the overall process is less than 60% by weight.

11. The process according to claim 1,
wherein
a degree of epimerization is less than 50% when R¹, R² and R³ are different from one another and are not COOH.

12. The process according to claim 1,
wherein
the product fraction is converted without further purification to conversion products.

13. The process according to claim 1,
further comprising one or more subsequent process for purification and isolation of the hydroxycarboxylic acid from the product fraction.

14. The process according to claim 1, wherein hydroxycarboxylic acids are α- and β-hydroxycarboxylic acids.

15. The process according to claim 12, wherein the conversion products are acrylic acid derivatives.
